(19) United States
(12) Patent Application Publication
(10) Pub. No.: US 2025/0319317 A1
(43) Pub. Date: Oct. 16, 2025

(54) PACING SYSTEM FOR WIRELESS EXTRA-CARDIAC PACING

(71) Applicant: Riccardo Cappato, Milan (IT)

(72) Inventor: Riccardo Cappato, Milan (IT)

(21) Appl. No.: 19/172,524

(22) Filed: Apr. 7, 2025

Related U.S. Application Data
(60) Provisional application No. 63/632,758, filed on Apr. 11, 2024.

(51) Int. Cl. A61N 1/378; A61N 1/362; A61N 1/372

(52) U.S. Cl. CPC A61N 1/3787; A61N 1/362; A61N 1/37205

(57) ABSTRACT

A pacing system (100) for wireless extra-cardiac pacing of a heart (303) of a patient (301). The pacing system (100) includes a generator (102), a transmitter (104), and an implant device (106). The generator (102) is configured to generate energy. The transmitter (104) is configured to transmit the energy generated by the generator (102). The implant device (106) including an expandable stent that is configured to be implanted in the heart (303) of the patient (301), the implant device (106) including a receiver (220) that is configured to receive the energy that is transmitted by the transmitter (220), the implant device including a transducer (222) that is configured to transduce the energy into a pacing stimulus that is configured to pace the heart (303).

### RELATED APPLICATION

This Application is related to and claims priority on U.S. Provisional Patent Application Ser. No. 63/632,758, filed on Apr. 11, 2024, and entitled “PACING SYSTEM FOR WIRELESS EXTRA-CARDIAC PACING.” To the extent permissible, the contents of U.S. Provisional Application Ser. No. 63/632,758 are incorporated in their entirety herein by reference.

### BACKGROUND

The normal, healthy heart has an electrical system that regulates the rate at which the heart beats. The electrical system controls the events that occur when the heart pumps blood. The electrical system also is called the cardiac conduction system. The cardiac conduction system is made up of three main parts: a) the sinoatrial (SA) node, located in the right atrium of the heart, b) the atrioventricular (AV) node, located on the interatrial septum close to the tricuspid valve, and c) the His-Purkinje system, located along the walls of the heart's ventricles.

A heartbeat is a complex series of events. These events take place inside and around the heart. A heartbeat is a single cycle in which the heart's chambers relax and contract to pump blood. This cycle includes the opening and closing of the inlet and outlet valves of the right and left ventricles of the heart. Each heartbeat has two basic parts: diastole and systole. During diastole, the atria and ventricles of the heart relax and begin to fill with blood. At the end of diastole, the heart's atria contract (atrial systole) and pump blood into the ventricles. The atria then begin to relax. The ventricles then contract (ventricular systole), pumping blood out of the heart.

Each heartbeat is set in motion by an electrical signal from within the heart muscle. Each heartbeat begins with a signal from the SA node in a normal, healthy heart. The signal is generated as the vena cava fills the heart's right atrium with blood. The signal spreads across the cells of the right and left atria. This signal causes the atria to contract. This action pushes blood through the open valves from the atria into both ventricles.

The signal arrives at the AV node near the ventricles. It slows instantly to allow the right and left ventricles to fill with blood. The signal is released and moves along a pathway called the bundle of His, located in the ventricles' walls. From the bundle of His, the signal fibers divide into left and right bundle branches through the Purkinje fibers. These fibers connect directly to the cells in the walls of the left and right ventricles. The signal spreads across the cells of the ventricle walls, and both ventricles contract. This pushes blood through the pulmonary valve (for the right ventricle) to the lungs and through the aortic valve (for the left ventricle) to the rest of the body. As the signal passes, the walls of the ventricles relax and await the next signal. This process continues over and over.

However, a variety of conditions can cause the electrical conduction system to act erroneously, causing abnormal heartbeats or arrhythmias. A common type of arrhythmias is bradyarrhythmias. Bradyarrhythmias occur if the heart rate is slower than normal. If the heart rate is too slow, not enough blood reaches the brain, causing fainting. A heart rate slower than 60 beats per minute is considered a bradyarrhythmia in adults. Some people usually have slow heart rates, especially people who are very physically fit. For them, a heartbeat slower than 60 beats per minute is not dangerous and does not cause symptoms. But in other people, serious diseases or other conditions may cause bradyarrhythmias. Heart attacks can cause bradyarrhythmias, conditions that harm or change the heart's electrical activity (such as an underactive thyroid gland or aging) or an imbalance of chemicals or other substances in the blood, to name a few.

Electric pacing is one strategy to treat patients with abnormal heartbeats or arrhythmias, particularly bradyarrhythmias. Electric pacing is sometimes achieved with a pacemaker. A pacemaker is a small battery-operated device that assists the heart to beat in a regular rhythm. The pacemaker can be inserted into the patient through a simple surgery using either a local anesthetic or a general anesthetic. In some cases, the pacemaker is inserted in the left shoulder area, where an incision is made below the collar bone, creating a small pocket where the generator is implanted in the patient's body. The right ventricular lead can be positioned away from the apex of the right ventricle and up on the interventricular septum, below the outflow tract, to prevent deterioration of the strength of the heart.

After the surgery, a follow-up session is conducted, during which the pacemaker is checked using a “programmer” that can communicate with the device and allows a healthcare professional to evaluate the system's integrity and determine the settings, such as pacing voltage output. The patient should have the strength of their heart frequently analyzed with echocardiography every 1 or 2 years to ensure that placement of the right ventricular lead has not weakened the left ventricle. Since a pacemaker uses batteries, the device itself will need replacement as the batteries lose power. Device replacement is usually a more straightforward procedure than the original insertion as it does not normally require leads to be implanted. The typical replacement requires surgery in which an incision is made to remove the existing device, the leads are removed from the existing device, the leads are attached to the new device, and the new device is inserted into the patient's body, replacing the previous device.

However, there are a variety of short-, intermediate-, and long-term complications that do not infrequently require lead and/or device removal and replacement. Example complications include infection (in the implantation pocket, along the leads, along the inner lining of the heart, along the heart valves, spreading systemically, etc.), swelling, bruising or bleeding at the generator site, damage to blood vessels or nerves near the device, and the presence of damaged or non-functioning leads, to name a few. In addition, occlusion of the access vein by the leads is not uncommon. However, lead extraction can also result in venous obstruction, along with other complications. Pacemakers sometimes include a canister attached to a wall of the right ventricle. This poses a severe risk of death if the canister becomes detached and embolized within the vascular circulation.

### SUMMARY

The present invention is directed toward a pacing system for wireless extra-cardiac pacing of a heart of a patient. In various embodiments, the pacing system includes a generator, a transmitter, and an implant device. The generator is configured to generate energy. The transmitter is configured to transmit the energy generated by the generator. The implant device including an expandable stent that is configured to be implanted in the heart of the patient, the implant device including a receiver that is configured to receive the energy that is transmitted by the transmitter, the implant device including a transducer that is configured to transduce the energy into a pacing stimulus that is configured to pace the heart.

In some embodiments, the generator including one of (i) a first energy source, (ii) a second energy source, and (iii) generator circuitry.

In certain embodiments, the first energy source and the second energy source each includes one of a power supply, a battery, a laser, a magnet, an emitter, a transducer, a light source, a waveform generator, and an induction coil.

In various embodiments, the implant device includes a device body that is at least partially formed from a mesh material.

In some embodiments, the implant device includes a device body that is configured to expand and retract.

In certain embodiments, the implant device includes a routing layer that is configured to interconnect the receiver, the transducer, and the electrode.

In various embodiments, the receiver is configured to one of capture, receive, and absorb the energy transmitted by the transmitter.

In some embodiments, the transducer is configured to transduce ultrasound energy into the pacing stimulus that paces the heart, the pacing stimulus including electrical energy.

In certain embodiments, the generator includes an energy source that provides energy for the generator.

In various embodiments, the energy source includes one of a power supply, a battery, a laser, a magnet, an emitter, a transducer, a light source, a waveform generator, a thermal source, a chemical source, and an induction coil.

The present invention is also directed toward a pacing system for wireless extra-cardiac pacing of a heart of a patient. In various embodiments, the pacing system includes a generator, a transmitter, and an implant device. The generator is configured to generate energy. The transmitter is configured to transmit the energy generated by the generator. The implant device including an expandable stent that is configured to be implanted in the heart of the patient, the implant device being configured to (i) receive the energy, (ii) transduce the energy into a pacing stimulus, and (iii) pace the heart with the pacing stimulus, the implant device including a receiver that is configured to one of capture, receive, and absorb the energy transmitted by the transmitter.

In certain embodiments, the implant device includes a transducer that is configured to transduce the energy transmitted from the transmitter into the pacing stimulus.

In various embodiments, the transducer is configured to transduce ultrasound energy into electrical power that paces the heart.

In some embodiments, the implant device includes an electrode that is configured to pace the heart with the pacing stimulus.

In certain embodiments, the implant device includes a device body that is at least partially formed from a mesh material.

In various embodiments, the implant device includes a device body that is configured to expand and retract.

In some embodiments, the generator includes an energy source that provides energy for the generator.

In certain embodiments, the energy source includes one of a power supply, a battery, a laser, a magnet, an emitter, a transducer, a light source, a waveform generator, a thermal source, a chemical source, and an induction coil.

In various embodiments, the transmitter is configured to wirelessly transmit the energy generated by the generator to the implant device.

The present invention is also directed toward a pacing system for wireless extra-cardiac pacing of a heart of a patient. In various embodiments, the pacing system includes a generator, a transmitter, and an implant device. The generator is configured to generate energy. The transmitter is configured to transmit the energy generated by the generator. The implant device including an expandable stent that is configured to be implanted in the heart of the patient. The device body including an expandable stent, the device body being at least partially formed from a mesh material, the implant device including a receiver that is configured to receive the energy that is transmitted by the transmitter, the receiver being configured to one of capture, receive, and absorb the energy transmitted by the transmitter, the implant device including a transducer that is configured to transduce the energy into a pacing stimulus, the pacing stimulus including electrical energy, the implant device including an electrode that is configured to pace the heart with the pacing stimulus, the implant device including a routing layer that is configured to interconnect the receiver, the transducer, and the electrode.

This summary is an overview of some of the teachings of the present invention and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic view of an embodiment of a pacing system for wireless extra-cardiac pacing of a heart of a patient, the pacing system having features of the present invention;

FIG. 2 is a simplified, partially transparent, perspective view of an embodiment of an implant device;

FIG. 3 is a simplified illustration of an embodiment of the pacing system for wireless extra-cardiac pacing of the heart of the patient, the pacing system being positioned within a portion of the patient, the pacing system including the implant device, a transmitter, and a generator;

FIG. 4 is a simplified illustration of an embodiment of a catheter positioned within a portion of the patient;

FIG. 5 is a simplified side view of an embodiment of the catheter coupled to the implant device and a balloon;

FIG. 6 is a simplified illustration of an embodiment of a portion of the pacing system for wireless extra-cardiac pacing of the heart of the patient, the pacing system being positioned within a portion of the heart, the pacing system including the implant device, a first catheter, a second catheter, and the balloon;

FIG. 7 is a simplified, partially transparent, perspective view of an embodiment of the implant device;

FIG. 8 is a simplified illustration of an embodiment of the implant device positioned within a portion of the heart, the implant device being shown in an expanded configuration;

FIG. 9 is a simplified illustration of the heart and a plurality of implant devices implanted in portions of the heart;

FIG. 10 is a simplified schematic view of an embodiment of a portion of the pacing system for wireless extra-cardiac pacing of a heart of a patient, the pacing system including the transmitter and the generator;

FIG. 11 is a simplified illustration of an embodiment of the pacing system for wireless extra-cardiac pacing of the heart of the patient, the pacing system being positioned within a portion of the patient, the pacing system including a plurality of implant devices, a transmitter, and a generator;

FIG. 12 is a flow chart outlining one embodiment of a method for wireless extra-cardiac pacing of the heart of the patient; and

FIG. 13 is a flow chart outlining yet another embodiment of a method for wireless extra-cardiac pacing of the heart of the patient.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Embodiments of the pacing systems, devices, and related methods disclosed herein are configured to enable wireless extra-cardiac pacing of a heart of a patient. In particular, a pacing system can be implanted within the patient so that the pacing system wirelessly paces the patient's heart. As used herein, the “heart” is understood to mean the heart including both atrial chambers, both ventricular chambers, the septum, the pulmonary veins, the coronary sinus, the fossa ovalis, the superior vena cava, the inferior vena cava, the muscular sleeves, the vascular walls, connected, electrically active tissues, and all other heart support structures in or near the heart.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention, as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming but would nevertheless be a routine undertaking of engineering for those of ordinary skill in the art having the benefit of this disclosure.

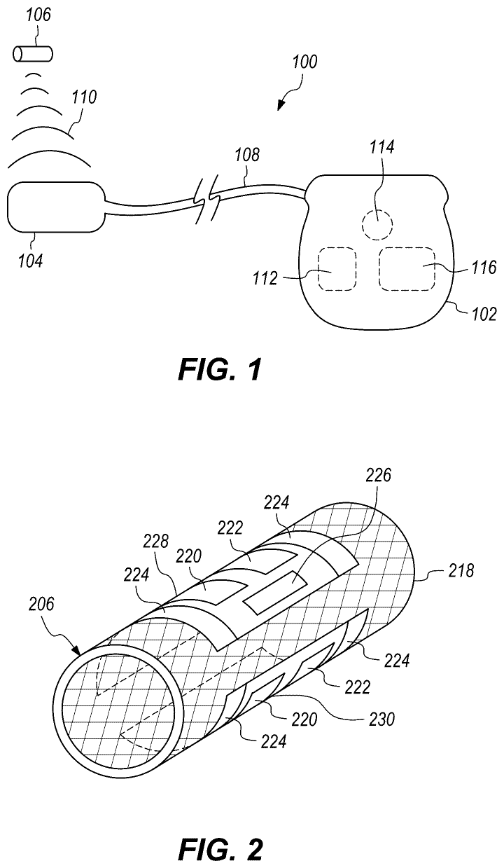

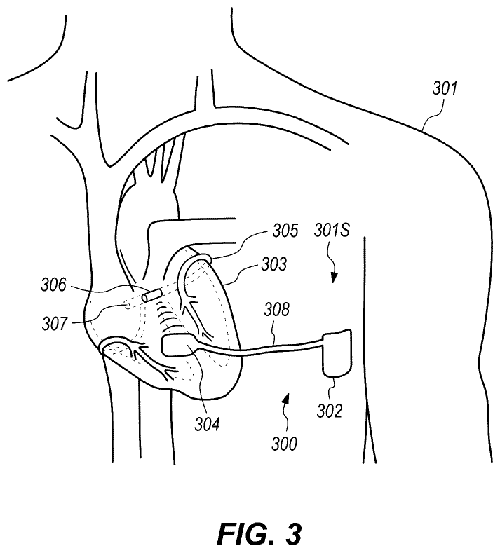

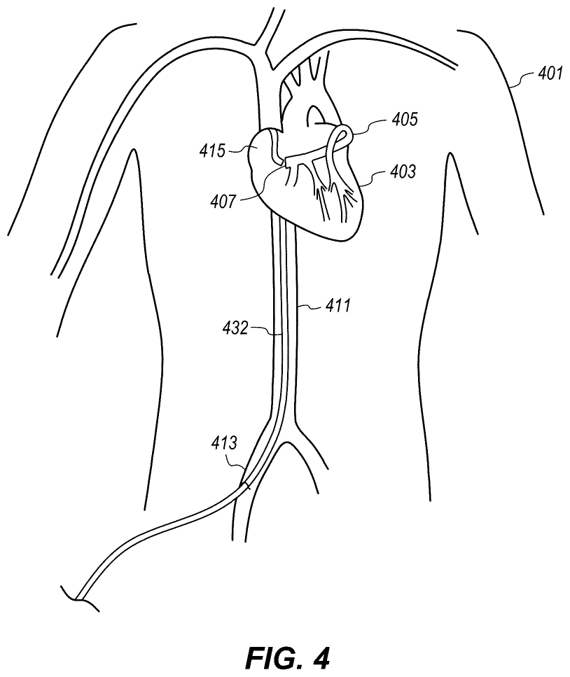

FIG. 1 is a simplified schematic view of an embodiment of a pacing system 100 for wireless extra-cardiac pacing of a heart 303 (for example, as illustrated in FIG. 3) of a patient 301 (for example, as illustrated in FIG. 3). The pacing system 100 can vary depending on its design requirements. It is understood that the pacing system 100 can include additional components, systems, subsystems, and elements other than those specifically shown and/or described herein. Additionally, or alternatively, the pacing system 100 can omit one or more of the components, systems, subsystems, and elements that are specifically shown and/or described herein. In some embodiments, various components of the pacing system 100 can be positioned in a different manner than what is specifically illustrated in FIG. 1. In certain embodiments, the pacing system 100 can include a generator 102, a transmitter 104, an implant device 106, and a coupler 108.

The generator 102 generates any suitable form of energy 110. Non-exclusive, non-limiting examples of energy 110 generated by the generator 102 include mechanical energy, chemical energy, thermal energy, nuclear energy, ultrasound, ultrasonic, chemical energy, electrical energy, magnetic energy, electromagnetic energy, elastic energy, gravitational energy, sound energy, and/or light energy. The generator 102 can be configured to be implanted within the patient 301.

The generator 102 can vary depending on its design requirements or the design requirements of the pacing system 100, the transmitter 104, the implant device 106, and the coupler 108. It is understood that the generator 102 can include additional components, systems, subsystems, and elements other than those specifically shown and/or described herein. Additionally, or alternatively, the generator 102 can omit one or more of the components, systems, subsystems, and elements that are specifically shown and/or described herein. In some embodiments, various components of the generator 102 can be positioned in a different manner than what is specifically illustrated in FIG. 1. In some embodiments, as illustrated in FIG. 1, the generator 102 can include a first energy source 112, a second energy source 114, and/or generator circuitry 116.

The transmitter 104 transmits the energy 110 generated by the generator 102 to the implant device 106. In some embodiments, the transmitter 104 can wirelessly transmit the energy 110. The transmitter 104 can transmit the energy 110 via any suitable transmission method known in the art.

The transmitter 104 can vary depending on its design requirements or the design requirements of the pacing system 100, the generator 102, and the implant device 106. It is understood that the transmitter 104 can include additional components, systems, subsystems, and elements other than those specifically shown and/or described herein. Additionally, or alternatively, the transmitter 104 can omit one or more of the components, systems, subsystems, and elements that are specifically shown and/or described herein. In some embodiments, various components of the transmitter 104 can be positioned in a different manner than what is specifically illustrated in FIG. 1. The transmitter 104 can be configured to be implanted within the patient 301.

The implant device 106 receives the energy 110 transmitted by the transmitter 104. The implant device 106 is configured to be implanted within the heart 303 of the patient 301. The implant device 106 is configured to transduce the energy 110 into a pacing stimulus for pacing the heart 303 of the patient 301.

The implant device 106 can vary depending on its design requirements or the design requirements of the pacing system 100, the generator 102, and the transmitter 104. It is understood that the implant device 106 can include additional components, systems, subsystems, and elements other than those specifically shown and/or described herein. Additionally, or alternatively, the implant device 106 can omit one or more of the components, systems, subsystems, and elements that are specifically shown and/or described herein. The implant device 106 can have a somewhat tubular, cylindrical, and/or prism shape.

The coupler 108 can couple the generator 102 to the transmitter 104. While the coupler 108 is shown as a physical coupling, it is appreciated that the coupling between the generator 102 and the transmitter 104 can be wireless.

The coupler 108 can vary depending on its design requirements or the design requirements of the pacing system 100, the generator 102, and the transmitter 104. It is understood that the coupler 108 can include additional components, systems, subsystems, and elements other than those specifically shown and/or described herein. Additionally, or alternatively, the coupler 108 can omit one or more of the components, systems, subsystems, and elements that are specifically shown and/or described herein. The coupler 108 can include energy guides, routing layers, wireless communicator systems, and/or any suitable coupling device or system.

The first energy source 112 and the second energy source 114 can generate energy 110 for the generator 102, either individually, or in combination. The energy sources 112, 114 can vary depending on the design requirements of the pacing system 100 and/or the generator 102. In some embodiments, the energy sources 112, 114 can include at least one of a power supply, a battery, a laser, a magnet, an emitter, a transducer, a light source, a waveform generator, a thermal source, a chemical source, and/or an induction coil.

The generator circuitry 116 can support communication between components of the pacing system 100, such as the generator 102 and the transmitter 104. The generator circuitry 116 can vary depending on its design requirements or the design requirements of the pacing system 100, the generator 102, and the transmitter 104. It is understood that the generator circuitry 116 can include additional components, systems, subsystems, and elements other than those specifically shown and/or described herein. Additionally, or alternatively, the generator circuitry 116 can omit one or more of the components, systems, subsystems, and elements that are specifically shown and/or described herein.

FIG. 2 is a simplified, partially transparent, perspective view of an embodiment of an implant device 206. As shown in FIG. 2, in some embodiments, the implant device 206 can include a device body 218, a receiver 220, a transducer 222, an electrode 224, device circuitry 226, a first routing layer 228, and/or a second routing layer 230.

The device body 218 can form the primary structure of the implant device 206. The device body 218 can have any suitable shape. The device body 218 can be configured to expand and/or retract. It may be appreciated that the device body 218 can be self-expanding. In some embodiments, the device body 218 is held under tension within a sheath or sleeve (not shown) covering device body 218. Retraction of the sheath (not shown) reveals the implant device 206, allowing the device body 218 to expand. It may also be appreciated that in some embodiments, the device body 218 is again collapsible to allow repositioning or removal if desired.

The device body 218 can be configured to be implanted within the patient 301 (as shown in FIG. 3) and/or the heart 303 (as shown in FIG. 3) without significantly or detrimentally occluding the blood flow of the patient 301. The device body 218 can vary depending on its design requirements and/or the design requirements of the pacing system 100 (as shown in FIG. 1) and/or the transmitter 104 (as shown in FIG. 1). The device body 218 can be at least partially formed from at least one of a mesh material, a metal, a polymer wire, a plastic, a natural material, and/or a synthetic material. The device body 218 can include a stent or a structure similar to a stent.

The device body 218 can include components and/or elements that can be configured to provide electrical signals and/or stimuli for pacing the heart 303. The receiver 220, the transducer 222, the electrode 224, the device circuitry 226, the first routing layer 228, and/or the second routing layer 230 can be integrally formed with or coupled to the device body 218. The device body 218 can be configured to retain the components and/or the elements of the implant device 206 within the heart 303 (in some embodiments, the coronary vasculature of the heart 303, such as against the luminal walls of the coronary vasculature). The device body 218 can be configured to provide mechanical radial support to the heart 303, similar to the functionality of a stent.

In some embodiments, the device body 218 can release an eluting drug over a period of time to counteract the pro-thrombotic and inflammatory potential of the implant device 206 at its deployed location. In other embodiments, one or more drug-eluting layers (not shown) can be coupled to the device body 218. In certain embodiments, the device body 218 can include multiple layers, including one or more drug-eluting layers and/or protective layers. In various embodiments, other components of the device body 218 (such as the first routing layer 228 and/or the second routing layer 230) can include an eluting drug and/or one or more drug-eluting layers. The device body 218 can include drugs such as immunosuppressive and antiproliferative drugs to counteract the pro-thrombotic and inflammatory potential of the device body 218. Specific non-limiting, non-exclusive drugs usable within the implant device 206 and/or the device body 218 include sirolimus, paclitaxel, and everolimus. However, it is appreciated that any suitable, elutable drug can be utilized within the implant device 206 and/or the device body 218.

The receiver 220 can communicate with the transmitter 104. In some embodiments, the receiver 220 can capture, receive, and/or absorb the energy 110 (as shown in FIG. 1) transmitted from the transmitter 104. The receiver 220 can vary depending on its design requirements and/or the design requirements of the pacing system 100, the transmitter 104, the implant device 206, and/or the device body 218. The receiver 220 can be positioned in any suitable position in the implant device 206, including those shown in FIG. 2. Although two receivers 220 are displayed in FIG. 2, it is appreciated that the implant device 206 can include any suitable number of receivers 220, including one receiver 220 or three or more receivers 220.

The transducer 222 can transduce the energy 110 transmitted from the transmitter 104. In certain embodiments, the transducer 222 transduces the energy 110 into an electrical signal and/or a pacing stimulus used to pace the heart 303. The transducer 222 can vary depending on its design requirements and/or the design requirements of the pacing system 100, the transmitter 104, the implant device 206, and/or the device body 218. The transducer 222 can be positioned in any suitable position in the implant device 206, including those shown in FIG. 2. Although two transducers 222 are displayed in FIG. 2, it is appreciated that the implant device 206 can include any suitable number of transducers 222, including one transducer 222 or three or more transducers 222. In some embodiments, the transducer 222 can transduce ultrasound energy into electrical power used to pace the heart 303.

The electrode 224 can be configured to deliver the electrical signal and/or pacing stimulus transduced by the transducer 222 to the heart 303. The electrical signal and/or pacing stimulus delivered by the electrode 224 can activate surrounding cardiac tissue, artificially pacing the heart 303. The electrode 224 can vary depending on its design requirements and/or the design requirements of the pacing system 100, the transmitter 104, the implant device 206, and/or the device body 218. The electrode 224 can be positioned in any suitable position in the implant device 206, including those shown in FIG. 2. Although four electrodes 224 are displayed in FIG. 2, it is appreciated that the implant device 206 can include any suitable number of electrodes 224, including one electrode 224 or two or more electrodes 224.

The device circuitry 226, the first routing layer 228, and/or the second routing layer 230 can enable transfers of energy and communication between the components of the implant device 206. In some embodiments, the components (220, 222, 224, 226) can be coupled to the first routing layer 228, and/or the second routing layer 230. However, in other embodiments, the components (220, 222, 224, 226) can be coupled or integrally formed with the device body 218. It may also be appreciated that in some embodiments, such as illustrated in FIG. 2, two sets of components (220, 222, 224, 226) are coupled on the device body 218, each set disposed on a routing layer 228, 230. The type and number of components (220, 222, 224, 226) may be the same or different on the first routing layer 228, and/or the second routing layer 230. Likewise, the first routing layer 228, and/or the second routing layer 230 may act independently or may be interrelated.

In some embodiments, the first routing layer 228, and/or the second routing layer 230 includes two electrodes 224, wherein each pair of electrodes 224 forms a bipole. Thus, an electric field can be generated between the electrodes 224. The shape of the electric field can be modified by the arrangement of the electrodes 224, such as by the distance between them. It may be appreciated that any number of electrodes 224 may be present, including a single electrode 224 acting in a unipolar fashion. Likewise, a plurality of electrodes 224 may be present wherein select electrodes are activated.

FIG. 3 is a simplified illustration of an embodiment of the pacing system 300 for wireless extra-cardiac pacing of the heart 303 of the patient 301, the pacing system 300 being positioned within a portion of the patient 301, the pacing system 300 including the generator 302, the transmitter 304, and the implant device 306. The pacing system 300 and the various components illustrated can be substantially similar in form and function as previously shown and described with respect to FIGS. 1-2.

In some embodiments, such as illustrated in FIG. 3, the generator 302 can be implanted in a subcutaneous space 301S within the torso of the patient 301, near the heart 303. The subcutaneous space 301S can include the subcutaneous thorax. The transmitter 304 can be implanted in front of the subcutaneous projection of the underlying proximal anterior coronary vein of the heart 303. In certain embodiments, the transmitter 304 can be implanted in the subcutaneous or submuscular left hemithorax, either separated from or incorporated with the generator 302.

In some embodiments, the transmitter 304 can be positioned in the left parasternal space, 1-2 cm away from the sternal midline. In other embodiments, the transmitter 304 is positioned at a short distance, such as less than or equal to 11 cm, from the implant device 306 to limit the energy dispersion across the tissue interposed between them. The implant device 306 can be implanted within a coronary vein 305 near the heart 303. In some embodiments, such as illustrated in FIG. 3, the implant device 306 is implanted near a coronary sinus 307, within the coronary vein 305. In other embodiments, the implant device 306 is implanted 1 to 3 cm distal to the coronary sinus 307. The generator 302 and the transmitter 304 can be coupled via a coupler 308. While the coupler 308 is shown as a physical coupling implanted within the patient 301, it is appreciated that the coupling between the generator 302 and the transmitter 304 can be wireless. Energy 110 (as illustrated in FIG. 1) from the generator 302 can be transmitted from the transmitter 304 to the implant device 306 within the coronary vein 305. Even though the coronary veins 305 are located outside of the heart 303, the coronary veins 305 are in anatomical contact with its epicardial surface. Therefore, pacing can have increased effectiveness when applied from any location inside the coronary veins 305. This pacing method can allow easier capture of the surrounding atrial and ventricular myocardium, usually at a low amplitude threshold. Variation in amplitude threshold of the electrical impulse delivered by the implant device 306 may be varied in the individual patient 301 depending on the location of the implant device 306 relative to the target myocardial tissue and on changes in pacing threshold occurring over time.

Wireless pacing can be achieved by the use of the pacing system 300. As mentioned previously, in certain embodiments, the generator 302 generates the energy 110, such as magnetic, ultrasound, radiofrequency, or a combination of any of these, which is transmitted from the transmitter 304 according to a pacing algorithm. The energy 110 is transduced into electric signals, and, therefore, pacing, which is provided to the patient 301. The generator 302 also includes generator circuitry 116 (as illustrated in FIG. 1) in support of the communications between the elements of the pacing system 300, including, in some embodiments, sensing when such energy 110 is needed. For example, in some embodiments, the generator circuitry 116 includes at least one detection algorithm, which is used to analyze sensed atrial and/or ventricular activity. Such activity is sensed by one or more electrodes 224 (as illustrated in FIG. 2) or sensors located at one or more locations within the pacing system 300, including on the generator 302, the transmitter 304, the implant device 306, or any combination of these elements.

In some embodiments, the routing layers 228, 230 (as illustrated in FIG. 2) can be arranged so as to sense atrial, ventricular, and/or electrophysiological activity from a particular area of the patient 301. For example, the implant device 306 can be positioned so that the first routing layer 228 faces an atrium and the second routing layer 230 faces a ventricle. Thus, the first routing layer 228 would be configured to sense atrial activity, and the second routing layer 230 would be configured to sense ventricular activity.

The sensed electrical activity correlates to the contractions of the heart 303. The heart 303 contracts due to signals from the electrical conduction system of the heart 303. Each beat begins with a signal from the SA node. Impulses originating from the SA node spread to adjacent myocardial cells and propagate as a wave of depolarization through the atria. This signal causes the atria to contract. The signal then arrives at the AV node near the ventricles. The signal spreads across the cells of the ventricle walls like a wave of depolarization, and both ventricles contract. As the signal passes, the walls of the ventricles relax and await the next signal. These changes in electric charge (depolarization/repolarization) are sensed to determine if the heart 303 is functioning properly or if the heart needs assistance in pacing.

The changes in electric charge can be visualized with an electrocardiogram (ECG). An ECG curve has distinctive characteristics depending on the location of the electrode recording it. If a wave of depolarization travels towards an electrode 224 attached to a positive input terminal of an ECG amplifier and away from the electrode 224 attached to the negative terminal, a positive-going deflection results. If the wave travels away from the positive electrode 224 towards the negative electrode 224, a negative-going deflection results. Thus, an ECG curve typically includes a P wave (indicating depolarization of the atrial muscles), a QRS complex (indicating depolarization of the ventricular muscles), and a T wave (restoration of electrical potential), the specifics of which may vary due to location of the sensing and recording. ECG curves generated from various locations in the body may provide different insights into the pacing activity of the heart 303.

In various embodiments, the pacing system 300 responds to irregularities in the natural pacing activity of the heart 303, and in other embodiments, the pacing system 300 provides continual regular pacing. In instances of intermittent arrhythmias, the pacing system 300 remains passive in terms of pacing when the heart 303 is regularly beating. If heartbeats are missing or irregular, the detection algorithm immediately determines the need for pacing and provides the requisite energy 110 to institute an artificial pacing stimulus by one or more electrodes 224. The one or more electrodes 224 stimulate the nearby cardiac tissue, leading to the desired contraction. In instances of continual arrhythmias, such as bradyarrhythmias or slow heartbeat, the pacing system 300 provides the requisite energy 110 for continued artificial pacing stimulus.

In certain embodiments, the elements of the pacing system 300 can be chosen and positioned in a variety of orientations so as to function as a single-chamber pacemaker, a dual-chamber pacemaker, and a biventricular pacemaker as non-limiting, non-exclusive examples. The pacing system 300 can provide single-chamber pacing to the right ventricle by positioning the implant device 306 within the coronary vein 305 and the transmitter 304 and generator 302 at a convenient distance from the implant device 306, such as in the subcutaneous left parasternal space. Upon receiving the energy 110, the implant device 306 stimulates the right ventricle to pace the heart 303.

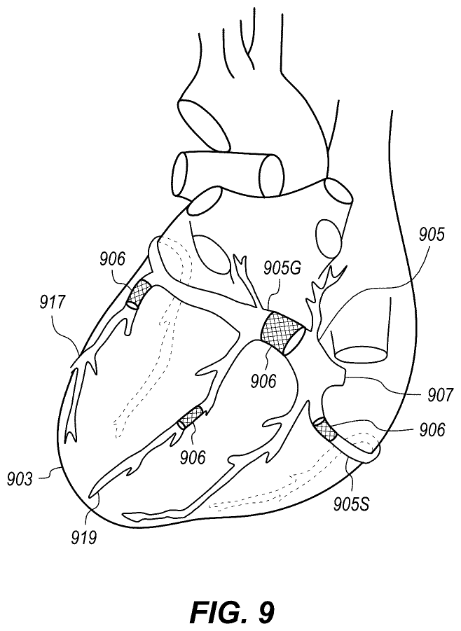

Depending on the patient's symptoms 301 and the type of pacing desired, the implant device 306 can be positioned to stimulate the right atrium to provide single-chamber pacing. In such instances, the implant device 306 may be positioned within the coronary vein 305 to provide stimulation to the nearby atrium. It may be appreciated that in some situations, single-site pacing within a chamber is insufficient, such as in the long-term prevention of atrial fibrillation. Therefore, in some embodiments, more than one implant device 306 is positioned within a coronary vein 305 near a particular chamber for stimulation thereto (such as shown in FIG. 9). In such embodiments, the transmitter 304 and attached generator 302 are located at a convenient distance from the implant devices 306, to optimize the efficiency of the pacing system 100 in limiting energy dispersion across the body tissues interposed therebetween. The transmitter 304 provides energy 110 to each of the implant devices 306 so as to provide appropriate pacing from multiple sites.

The pacing system 300 can also provide dual-chamber pacing by positioning the implant device 306 near the right atrium by positioning a first implant device 306 within the coronary vein 305 and positioning a second implant device 306 near the right ventricle by positioning the second implant device 306 within the coronary vein 305. Again, the transmitter 304 and the generator 302 can be positioned at a convenient distance from the implant devices 318, such as in the subcutaneous left parasternal space. Upon receiving the energy 110, the implant device 318 can be configured to stimulate the right atrium and ventricle to pace the heart 303.

In some embodiments, the pacing system 300 can provide biventricular pacing. Before implantation of the pacing system 300, imaging can be used to identify vital areas embedded into the desynchronized tissue of the patient's heart 303. Non-limiting, non-exclusive examples of pre-implant imaging examinations include positron emission tomography (PET), thallium myocardial scintigraphy, technetium myocardial scintigraphy, and stress echocardiogram. Based on imaging data, the selection of the target area or areas for implantation of the implant device 306 can be planned ahead of implantation.

In one embodiment, biventricular pacing is provided by positioning a first implant device 306 near the right atrium by positioning the implant device 306 within the coronary vein 305 and positioning a second implant device 306 near the right ventricle by positioning the second implant device 306 within the coronary vein 305. Placement of the implant devices 306 at target areas within the coronary vasculature would allow testing, through a true multi-site resynchronization pacing, the ability to improve left ventricular contraction by optimizing recruitment of all available tissue from the impaired left ventricle.

FIG. 4 is a simplified illustration of an embodiment of a first catheter 432 positioned within a portion of the patient 401. FIG. 4 illustrates the advancement of the first catheter 432 through a femoral vein 413, along an abdominal vena cava 411, and into a right atrium 415 of the heart 403 of the patient 401. The first catheter 432 can be advanced by a guidewire (not shown) that is passable through the first catheter 432. Any suitable guidewire (not shown) can be used to advance the catheters described herein, including guidewires that are thin, flexible wires that can be inserted into a confined or tortuous space to act as a guide for subsequent insertion of a stiffer or bulkier instrument. In some embodiments, the first catheter 432 can be advanced over the guidewire along the path created by the guidewire. The first catheter 432 can be additionally advanced sufficiently within the coronary sinus 407 to provide a pathway to any suitable implantation location for the implant device 306 (as illustrated in FIG. 3) within the coronary vein 405, near the coronary sinus 407.

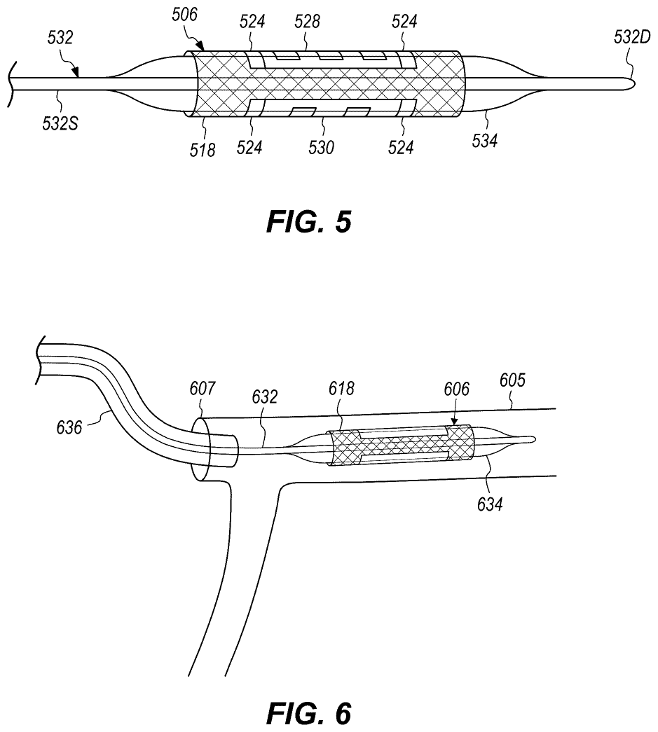

FIG. 5 is a simplified side view of an embodiment of the first catheter 532 coupled to the implant device 506 (including the device body 518, electrodes 524, the first routing layer 528, and the second routing layer 530) and a balloon 534. The implant device 506 is implantable within the patient 401 (as illustrated in FIG. 4) with a delivery system, such as the first catheter 532. FIGS. 4-6 illustrate embodiments of a delivery system and method of delivering the implant device 506 within the body of the patient 401. The delivery system can include the first catheter 532, which can be used to advance the implant device 506 to the coronary sinus 407 (as illustrated in FIG. 4) for implantation into the coronary vein 405 (as illustrated in FIG. 4).

The first catheter 532 can vary depending on its design requirements or the design requirements of the implant device 506. The first catheter 532 can include a tubular shaft 532S that can be flexible and a catheter distal end 532D that can be formed into a shape, such as a dome, to assist in accessing the coronary sinus 407. In some embodiments, the first catheter 532 can include an Amplatz coronary catheter. As shown in FIG. 5, the implant device 506 can be coupled to the first catheter 532 for delivery to any suitable implant location within the patient 401.

The balloon 534 can be inflatable. In some embodiments, the balloon 534 can also be coupled to the first catheter 532 near the catheter distal end 532D. The implant device 506 can be coupled to the balloon 534 so that the implant device 506 at least partially encircles the balloon 534. The balloon 534 can be movable between a deflated state and an inflated state. In the deflated state, the balloon 534 can have a reduced circumference so that the balloon 534 can be advanced through portions of the patient 401. In the inflated state, the balloon 534 can be inflated so that the implant device 506 is at least partially expanded and/or engages a portion of the patient 401. The balloon 534 can vary depending on its design requirements and/or the design requirements of the implant device 506 and/or the first catheter 532.

Before delivery, in some embodiments, the balloon 534 is moved to the deflated state, and the implant device 506 is coupled to the balloon 534 in a retracted configuration. In these configurations, the implant device 506 and the balloon 534 have a reduced circumference, minimizing the diameter for ease of advancement to the desired implantation location.

FIG. 6 is a simplified illustration of an embodiment of a portion of the pacing system 100 (illustrated in FIG. 1) for wireless extra-cardiac pacing of the heart 403 (illustrated in FIG. 4) of the patient 401 (illustrated in FIG. 4), the pacing system 100 being positioned within a portion of the heart 401, the pacing system 100 including the implant device 606, the first catheter 632, a second catheter 636, and the balloon 634. As illustrated in FIG. 6, the first catheter 632 can be advanced through the second catheter 636, into the coronary vein 605 for positioning therein. Once the implant device 606 is positioned within the coronary vein 605 near the coronary sinus 607, the balloon 634 can be inflated, such as with a fluid and/or a gas, to expand the implant device 606 so that the device body 618 contacts the inner walls of the coronary vein 605.

The second catheter 636 can vary depending on its design requirements and/or the design requirements of the implant device 606, the first catheter 632, and/or the balloon 634. The second catheter 636 can function similarly to an access catheter, and the first catheter 632 can function similarly to a delivery catheter, or vice versa. The use of “first” and “second” catheters 632, 636 are merely for ease of understanding, and the systems, and methods can utilize any suitable number of catheters, and devices for wireless extra-cardiac pacing, as described herein. Similarly, the use of “first” and “second” is merely demonstrative as placeholders, and it is understood that the “first” catheter can also be the “second,” “third,” “fourth,” etc. catheter in other contexts.

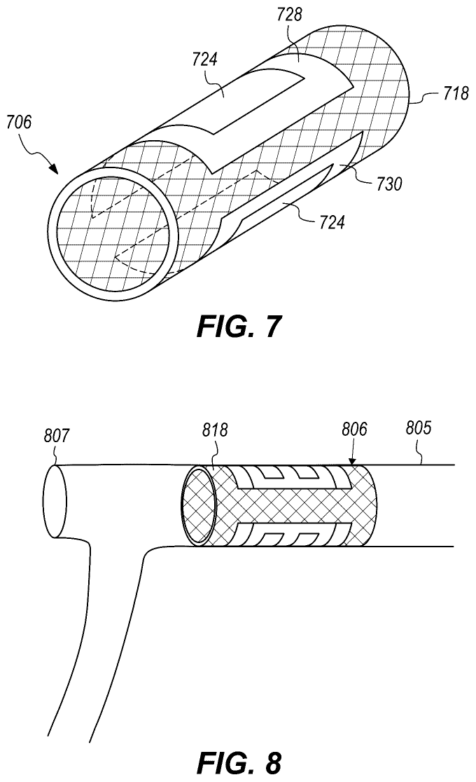

FIG. 7 is a simplified, partially transparent, perspective view of an embodiment of the implant device 706. The implant device 706 illustrated in FIG. 7 can be somewhat similar to other embodiments described herein. In FIG. 7, the implant device 706 is shown in an expanded state. The device body 718 in the embodiment of FIG. 7 includes a cylindrical, mesh-like material that is partially transparent. In some such embodiments, the first routing layer 728, and the second routing layer 730 can be coupled to the device body 718. In some embodiments, the first routing layer 728 and the second routing layer 730 can each include one electrode 724.

FIG. 8 is a simplified illustration of an embodiment of the implant device 806 positioned within a portion of the heart 401 (as illustrated in FIG. 4), the implant device 806 being shown in an expanded configuration. In FIG. 8, the implant device 818 is in contact with at least a portion of the coronary vein 805, near the coronary sinus 807. After the balloon 634 (as illustrated in FIG. 6) is moved to the deflated state and the first catheter 632 (as illustrated in FIG. 6) is removed from the interior of the coronary vein 805, the implant device 806 remains in the interior of the coronary vein 805.

FIG. 9 is a simplified illustration of the heart 903 and a plurality of implant devices 906 implanted in portions of the heart 903. In some embodiments, the pacing system 100 (as illustrated in FIG. 1) can include more than one implant device 906. In such embodiments, the implant devices 906 are implanted in various locations within the coronary vasculature. In particular, FIG. 9 illustrates a posteroinferior view of the heart 903 wherein the plurality of implant devices 906 were implanted at various locations within the coronary vein 905. In this non-exclusive, non-limiting example, individual implant devices 906 are implanted in a left marginal vein 917, a left posterior ventricular vein 919, a small coronary vein 905S, and the great coronary vein 905G, near the coronary sinus 907. This can be achieved by maintaining the second catheter 634 (as illustrated in FIG. 6) in place while delivering each implant device 906 through the use of the first catheter 632 (as illustrated in FIG. 6). Once each of the implant devices 906 is delivered, the second catheter 634 may be removed.

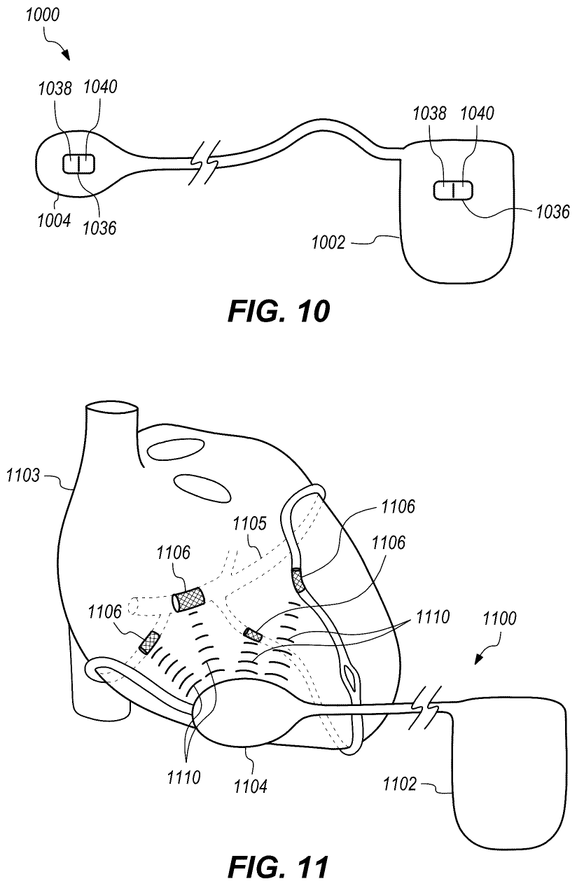

FIG. 10 is a simplified schematic view of an embodiment of a portion of the pacing system 1000 for wireless extra-cardiac pacing of a patient's heart, the pacing system 1000 including the generator 1002 and the transmitter 1004. In some embodiments, each of the generator 1002 and the transmitter 1004 can include a sensor assembly 1036 that includes a first sensor 1038 and a second sensor 1040. It is appreciated that each electrode 724 (for example, as illustrated in FIG. 7) may act as the sensor assembly 1036, the first sensor 1038, and/or the second sensor 1040.

FIG. 10 illustrates an embodiment wherein the transmitter 1004 includes a sensor assembly 1036 that includes a first sensor 1038 and a second sensor 1040, and the generator 1002 includes sensor assembly 1036 that includes a first sensor 1038 and a second sensor 1040. The sensor assembly 1036, the first sensor 1038, and/or the second sensor can be utilized in various unipolar and/or bipolar combinations. For example, the first sensor 1038 included in the transmitter 1004 may be used alone in a unipolar configuration. Likewise, the second sensor 1040 included in the transmitter 1004 may be used alone in a unipolar configuration. Similarly, the first sensor 1038 included in the generator 1002 may be used alone in a unipolar configuration. Further, the second sensor 1040 included in the generator 1002 may be used alone in a unipolar configuration.

In other embodiments, the first sensor 1038 may be used in combination with the second sensor 1040 in the sensor assembly 1040, both included in the transmitter 1004, in a bipolar configuration. The first sensor 1038 included in the transmitter 1004 can be used in combination with the first sensor 1038 included in the generator 1002, in a bipolar configuration. The first sensor 1038 included in the transmitter 1004 may be used in combination with the second sensor 1040 included in the generator 1002, in a bipolar configuration. The second sensor 1040 included in the transmitter 1004 may be used in combination with the first sensor 1038 included in the generator 1002, in a bipolar configuration. The second sensor 1040 included in the transmitter 1004 may be used in combination with the second sensor 1040 included in the generator 1002, in a bipolar configuration. The first sensor 1038 may be used in combination with the second sensor 1040, both included within the sensor assembly 1036 in the generator 1004, in a bipolar configuration.

The sensor assembly 1036, the first sensor 1038, and/or the second sensor 1040 can vary depending on their design requirements or the design requirements of the pacing system 1000, the generator 1002, and the transmitter 1004. It is understood that the sensor assembly 1036, the first sensor 1038, and/or the second sensor 1040 can include additional components, systems, subsystems, and elements other than those specifically shown and/or described herein. Additionally, or alternatively, the sensor assembly 1036, the first sensor 1038, and/or the second sensor 1040 can omit one or more of the components, systems, subsystems, and elements that are specifically shown and/or described herein. In some embodiments, various components of the sensor assembly 1036, the first sensor 1038, and/or the second sensor 1040 can be positioned in a different manner than what is specifically illustrated in FIG. 10.

In certain embodiments, each combination of sensor assembly 1036 and the sensors 1038, 1040 can provide a different view of the heart 903 (as illustrated in FIG. 9). One view of the heart 903 provides more precise electrical signals to assist the detection algorithm in the generator 1002 in differentiating between true and false cardiac electrical signals. In some embodiments, by providing more sensor assemblies 1036 and sensors 1038, 1040, the pacing system 1000 can increase discrimination power by providing more data to the detection algorithm and higher accuracy in reading the underlying cardiac signals. It may also be appreciated that one or more sensor assemblies and sensors 1036, 1038, 1040 may be included on the implant device 906 (as illustrated in FIG. 9) to add additional combinations and views of the heart 903.

It may be appreciated that the pacing system 1000 can be configured to communicate and function in coordination with a subcutaneous implantable defibrillator (not shown). The subcutaneous implantable defibrillator can be used to protect patients at risk of sudden cardiac death from life-threatening spontaneous ventricular arrhythmias but is not provided with any pacing option. In some embodiments, the pacing system 1000 is integrated with the implantable defibrillator to provide a combined pacing system that encompasses all possible physiological requirements of a sick heart 903. In a combined pacing system of the type indicated herein, the shock option for acute termination of ventricular fibrillation or fast ventricular tachycardia (provided by the implantable defibrillator) is integrated with anti-tachycardia pacing and anti-bradycardia pacing options (provided by the pacing system 1000). Such integration includes algorithms for coordinated function and communication mechanisms between the devices to achieve these goals.

FIG. 11 is a simplified illustration of an embodiment of the pacing system 1100 for wireless extra-cardiac pacing of the heart 1103 of the patient 401 (as illustrated in FIG. 4), the pacing system 1100 being positioned within a portion of the patient 401, the pacing system 1100 including a generator 1102, a transmitter 1104, and a plurality of implant devices 1106. FIG. 11 illustrates an anterior view of the heart 1103 wherein each of the plurality of implant devices 1106 are implanted at various locations within the coronary vein 1105, and a transmitter 1104 is positioned in communication with the implant devices 1106.

In certain embodiments, the generator 1102 and the transmitter 1104 can be positioned at varying distances from each of the plurality of implant devices 1106, such as in the subcutaneous left parasternal space, in order to optimize the efficiency of the pacing system 1100 by limiting energy dispersion across the body tissues interposed therebetween. The distance between the transmitter 1104 and each of the plurality of implant devices 1106 can depend on the form of energy 1100 to be used. However, in various embodiments, the distance between the transmitter 1104 and each of the plurality of implant devices 1106 can be greater than approximately 1 centimeter and less than approximately 10 centimeters. In other embodiments, the distance between the transmitter 1104 and each of the plurality of implant devices 1106 can be less than 1 centimeter and/or greater than 10 centimeters.

In the embodiment illustrated in FIG. 11, the transmitter 1104 provides energy 1110 to each of the implant devices 1106 so as to provide pacing from multiple sites, particularly for patients suffering from congestive heart failure. In certain embodiments, the energy 1110 is delivered by the transmitter 1104 in time intervals reproducing physiological beat-to-beat intervals in the normal heart as enabled by pacing algorithms allocated in the generator 1102.

For multisite pacing, single-beat energy pulses can be delivered by the transmitter 1104 to each of the plurality of implant devices 1106 at various times separated by a few milliseconds from each other to enable programmable time intervals of single-beat multisite activation that improve optimization of resynchronized contraction of a failing heart. Different combinations of multisite sequential cardiac activation through each of the plurality of implant devices 1106 can be programmed utilizing a central programming unit localized in the generator 1102.

In some embodiments, the transmitter 1104 transmits energy 1110 to each of the implant devices 1106 so as to provide pacing from multiple sites, in some instances, for patients suffering from congestive heart failure. It may be appreciated that in some embodiments, the energy 1110 is delivered by the transmitter 14 in time intervals reproducing physiological beat-to-beat intervals in the normal heart as enabled by pacing algorithms allocated in the generator 1102. For multisite pacing, single-beat energy pulses can be delivered by the transmitter 1104 to the implant device 1106 at various times, separated by a few milliseconds from each other to enable programmable time intervals of single-beat multisite activation that improve resynchronized contraction of a failing heart. Different combinations of multisite sequential cardiac activation through multiple implant device 1106 can be programmed by a central programming unit (not shown) localized in the generator 1102.

In some embodiments, the implant device 1106 is implanted in a portion of the vascular system outside of the coronary vasculature. For example, in some embodiments, at least one implant device 1106 is implanted within the superior vena cava, such as at the entrance of the superior vena cava into the right atrium. Such a position is reasonably close to the Sino-atrial node, where the atrial electrical impulse originated during physiological activation of the heart 1103. Again, the transmitter 1104 and the generator 1102 are positionable at a suitable distance from the implant device 1106 and provide energy 1110 thereto in a manner consistent with such positioning.

It may also be appreciated that energy 1110 may be delivered from one or more implant devices 1106 to the transmitter 1104 and/or generator 1102 or each other. In some embodiments, the energy 1110 includes sensing information that is transferred from the implant devices 1106 to the transmitter 1104 and/or generator 1102. In other embodiments, the energy 1110 includes inter-structure communication wherein energy communication is transferred between the implant devices 1106. For example, in some embodiments, two implant devices 1106 are implanted at a close distance from each other and work as unipolar elements to enable large base bipolar pacing using each implant device 1106 as one element of the bipole.

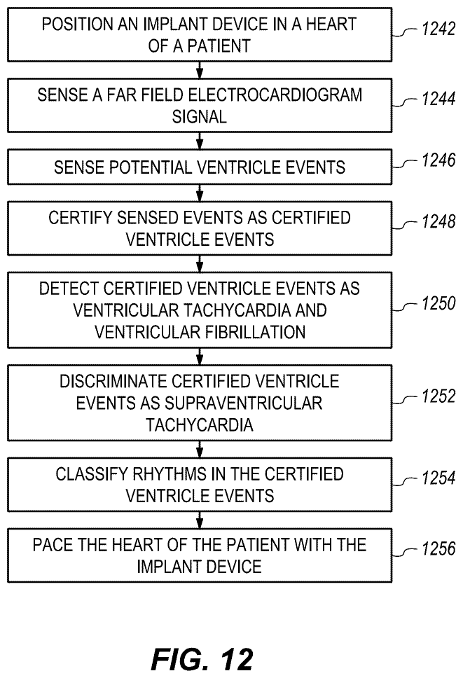

FIG. 12 is a flow chart outlining one embodiment of a method for wireless extra-cardiac pacing of a heart of a patient. It is understood that the method pursuant to the disclosure herein can include greater or fewer steps than those shown and described relative to FIG. 12. The method can omit one or more steps illustrated in FIG. 12. The method can add additional steps not shown and described in FIG. 12, and still fall within the purview of the present invention. Further, the sequence of the steps can be varied from those shown and described relative to FIG. 12. The sequence of steps illustrated in FIG. 12 is not intended to limit the sequencing of steps in any manner, and it is understood that the steps illustrated in FIG. 12 can be completed in any order.

In the embodiment illustrated in FIG. 12, at step 1242, an implant device is positioned within a heart of a patient. The implant device can include a device body, a receiver, a transducer, an electrode, device circuitry, a first routing layer, and/or a second routing layer. However, other designs of implant devices can be utilized by the methods described herein.

At step 1244, a far field electrocardiogram signal is sensed with the implant device.

At step 1246, potential ventricular events are sensed in the far field electrocardiogram signal.

At step 1248, the sensed ventricular events are certified as certified ventricular events.

At step 1250, the certified ventricular events are detected as ventricular tachycardia and ventricular fibrillation.

At step 1252, the certified ventricular events are discriminated as supraventricular tachycardia.

At step 1254, rhythms are classified in the certified ventricular events.

At step 1256, the heart of the patient is paced with the implant device based on the certified ventricular events.

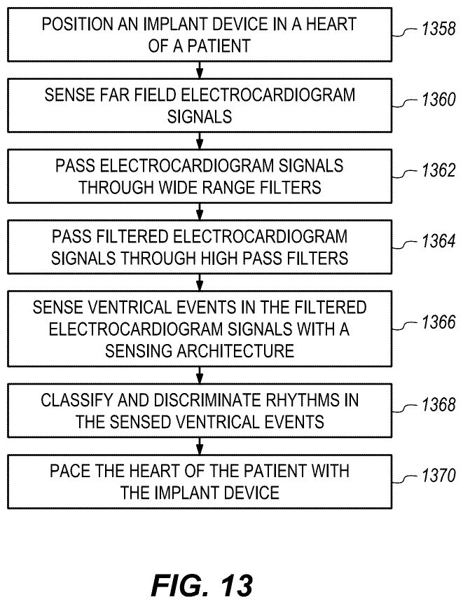

FIG. 13 is a flow chart outlining yet another embodiment of a method for wireless extra-cardiac pacing of a heart of a patient. It is understood that the method pursuant to the disclosure herein can include greater or fewer steps than those shown and described relative to FIG. 13. The method can omit one or more steps illustrated in FIG. 13. The method can add additional steps not shown and described in FIG. 13, and still fall within the purview of the present invention. Further, the sequence of the steps can be varied from those shown and described relative to FIG. 13. The sequence of steps illustrated in FIG. 13 is not intended to limit the sequencing of steps in any manner, and it is understood that the steps illustrated in FIG. 13 can be completed in any order.

In the embodiment illustrated in FIG. 13, at step 1358, an implant device is positioned within a heart of a patient. The implant device can include a device body, a receiver, a transducer, an electrode, device circuitry, a first routing layer, and/or a second routing layer. However, other designs of implant devices can be utilized by the methods described herein.

At step 1360, far field electrocardiogram signals are sensed with the implant device.

At step 1362, the far field electrocardiogram signals are passed through wide range filters.

At step 1364, the far field electrocardiogram signals are passed through high-pass filters.

At step 1366, ventricular events are sensed in the filtered far field electrocardiogram signals with a sensing architecture.

At step 1368, rhythms are classified and discriminated in the sensed ventricular events.

At step 1370, the heart of the patient is paced with the implant device based on the certified ventricular events.

It should be noted that, as used in this specification and the appended claims, the singular forms “a,” “an,” and “the” include plural referents unless the content and/or context clearly dictates otherwise. It should also be noted that the term “or” is generally employed in its sense, including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may be issued from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the detailed description provided herein. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of pacing systems and methods have been illustrated and described herein, one or more features of any one embodiment can be combined with one or more features of one or more of the other embodiments, provided that such combination satisfies the intent of the present invention.

While a number of exemplary aspects and embodiments of the pacing system and methods have been discussed above, those of skill in the art will recognize certain modifications, permutations, additions, and sub-combinations thereof. It is, therefore, intended that the following appended claims and claims hereafter introduced are interpreted to include all such modifications, permutations, additions, and sub-combinations as are within their true spirit and scope, and no limitations are intended to the details of construction or design herein shown.

What is claimed is:

1. A pacing system for wireless extra-cardiac pacing of a heart of a patient, the pacing system comprising:
a generator that is configured to generate energy;
a transmitter that is configured to transmit the energy generated by the generator; and
an implant device including an expandable stent that is configured to be implanted in the heart of the patient, the implant device including a receiver that is configured to receive the energy that is transmitted by the transmitter, the implant device including a transducer that is configured to transduce the energy into a pacing stimulus that is configured to pace the heart.

2. The pacing system of claim 1 wherein the generator includes one of (i) a first energy source, (ii) a second energy source, and (iii) generator circuitry.

3. The pacing system of claim 2 wherein the first energy source and the second energy source each includes one of a power supply, a battery, a laser, a magnet, an emitter, a transducer, a light source, a waveform generator, and an induction coil.

4. The pacing system of claim 1 wherein the implant device includes a device body that is at least partially formed from a mesh material.

5. The pacing system of claim 1 wherein the implant device includes a device body that is configured to expand and retract.

6. The pacing system of claim 1 wherein the implant device includes a routing layer that is configured to interconnect the receiver, the transducer, and the electrode.

7. The pacing system of claim 1 wherein the receiver is configured to one of capture, receive, and absorb the energy transmitted by the transmitter.

8. The pacing system of claim 1 wherein the transducer is configured to transduce ultrasound energy into the pacing stimulus that paces the heart, the pacing stimulus including electrical energy.

9. The pacing system of claim 1 wherein the generator includes an energy source that provides energy for the generator.

10. The pacing system of claim 9 wherein the energy source includes one of a power supply, a battery, a laser, a magnet, an emitter, a transducer, a light source, a waveform generator, a thermal source, a chemical source, and an induction coil.

11. A pacing system for wireless extra-cardiac pacing of a heart of a patient, the pacing system comprising:
a generator that is configured to generate energy;
a transmitter that is configured to transmit the energy generated by the generator; and
an implant device including an expandable stent that is configured to be implanted in the heart of the patient, the implant device being configured to (i) receive the energy, (ii) transduce the energy into a pacing stimulus, and (iii) pace the heart with the pacing stimulus, the implant device including a receiver that is configured to one of capture, receive, and absorb the energy transmitted by the transmitter.

12. The pacing system of claim 11 wherein the implant device includes a transducer that is configured to transduce the energy transmitted from the transmitter into the pacing stimulus.

13. The pacing system of claim 12 wherein the transducer is configured to transduce ultrasound energy into electrical power that paces the heart.

14. The pacing system of claim 11 wherein the implant device includes an electrode that is configured to pace the heart with the pacing stimulus.

15. The pacing system of claim 11 wherein the implant device includes a device body that is at least partially formed from a mesh material.

16. The pacing system of claim 11 wherein the implant device includes a device body that is configured to expand and retract.

17. The pacing system of claim 11 wherein the generator includes an energy source that provides energy for the generator.

18. The pacing system of claim 17 wherein the energy source includes one of a power supply, a battery, a laser, a magnet, an emitter, a transducer, a light source, a waveform generator, a thermal source, a chemical source, and an induction coil.

19. The pacing system of claim 11 wherein the transmitter is configured to wirelessly transmit the energy generated by the generator to the implant device.

20. A pacing system for wireless extra-cardiac pacing of a heart of a patient, the pacing system comprising:
a generator that is configured to generate energy;
a transmitter that is configured to transmit the energy generated by the generator; and
an implant device including a device body that is configured to be implanted in the heart of the patient, the device body including an expandable stent, the device body being at least partially formed from a mesh material, the implant device including a receiver that is configured to receive the energy that is transmitted by the transmitter, the receiver being configured to one of capture, receive, and absorb the energy transmitted by the transmitter, the implant device including a transducer that is configured to transduce the energy into a pacing stimulus, the pacing stimulus including electrical energy, the implant device including an electrode that is configured to pace the heart with the pacing stimulus, the implant device including a routing layer that is configured to interconnect the receiver, the transducer, and the electrode.
